(19) United States
(12) Patent Application Publication
(10) Pub. No.: US 2013/0164813 A1
(43) Pub. Date: Jun. 27, 2013

(54) Method for Inhibiting Transglutaminase 2 Activity Using 2-alkyloxy-3-phenylethynyl-4a,5-dihydropyrido[2,3-b]pyrazine Derivatives

(71) Applicants: Young Dae Gong, Seoul (KR); Soo Youl Kim, Gyeonggi-do (KR)

(72) Inventors: Young Dae Gong, Seoul (KR); Soo Youl Kim, Gyeonggi-do (KR)

(73) Assignee: DONGGUK UNIVERSITY INDUSTRY-ACADEMIC COOPERATION FOUNDATION, Seoul (KR)

(21) Appl. No.: 13/334,432

(22) Filed: Dec. 22, 2011

(51) Int. Cl. C12N 9/99

(52) U.S. Cl. 435/184

(57) ABSTRACT

The present invention relates to a method for inhibiting transglutaminase 2 activity using 2-alkyloxy-3-phenylethynyl-4a,5-dihydropyrido[2,3-b]pyrazine derivatives.

### CROSS-REFERENCE TO RELATED APPLICATIONS

None.

### STATEMENT OF FEDERALLY FUNDED RESEARCH

None.

### INCORPORATION-BY-REFERENCE OF MATERIALS FILED ON COMPACT DISC

None.

### TECHNICAL FIELD OF THE INVENTION

The present invention relates to a method for inhibiting transglutaminase 2 activity comprising contacting cells with 2-alkyloxy-3-phenylethynyl-4a,5-dihydropyrido[2,3-b]pyrazine derivatives.

### BACKGROUND OF THE INVENTION

In general, most chemotherapeutic agents are considerably toxic and cannot selectively kill cancer cells. Thus, there is an urgent need to develop new classes of anticancer agents that have low toxicity, but exhibit preventive and therapeutic effects before and after cancer occurrence.

Chemotherapy, is one of the methods for treating such malignant tumors, excluding surgery and radiation therapy, is generally called an anti-cancer agent, and most of the anti-cancer agents are substances that mainly inhibit nucleic acid synthesis to exhibit anti-cancer activity. Chemotherapy is largely divided into antimetabolites, alkylating agents, antimitotic drugs, hormones or the like. The antimetabolites inhibit the metabolism needed for the proliferation of cancer cells, and examples thereof include folic acid derivatives (methotrexate), purine derivatives (6-mercaptopurine, 6-thioguanine), and pyrimidine derivatives (5-fluorouracil and cytarabine). The alkylating agents exhibit anti-cancer effects by introducing alkyl groups into guanine bases of the DNA to modify a DNA structure and cleave a DNA chain, and examples thereof include nitrogen mustard compounds (chlorambucil and cyclophosphamide), ethyleneimine compounds (thiotepa), alkylsulfonate compounds (busulfan), nitrosourea compounds (carmustine), and triazine compounds (dacarbazine). The antimitotic drugs are cell cycle-specific drugs that block mitosis to inhibit cell division, and examples thereof include anti-cancer agents such as actinomycin D, doxorubicin, bleomycin, and mitomycin; plant alkaloids such as vincristine, vinblastine; and an antimitotic agent, taxane ring-containing taxoid. In addition, other anti-cancer agents include hormones such as adrenal cortical hormone and progesterone, and platinum-containing compounds such as cisplatin.

The biggest problem in chemotherapy is drug-resistance, which is the main reason the treatment eventually fails, despite initial success of therapy with anti-cancer agents.

Therefore, recent studies have focused on the development of anti-cancer agents with a novel chemical structure and new mechanisms of action. The present inventors discovered transglutaminase 2 (TGase 2) as a new target for cancer treatment, and the present inventors have revealed anti-cancer efficacy and its mechanism by inhibition of transglutaminase 2.

The present inventor and other research groups demonstrated that when the expression of transglutaminases was suppressed in chemoresistant breast cancer cells, the cancer cells were getting highly susceptible to chemicals, and finally died (Antonyak et al., Augmentation of tissue transglutaminase expression and activation by epidermal growth factor inhibit doxorubicin-induced apoptosis in human breast cancer cells. J Biol. Chem. Oct. 1, 2004; 279(40):41461-7; Dae-Seok Kim et al. Reversal of Drug Resistance in Breast Cancer Cells by Transglutaminase 2 Inhibition and Nuclear Factor-KB Inactivation. Cancer Res. Nov. 15, 2006; 66(22):10936-43s). Also, there is a strong reason for suppressing the activity of transglutaminases as the etiological mechanism for which the activation of transglutaminases is responsible is elucidated at the molecular level (Key Chung Park, Kyung Cheon Chung, Yoon-Seong Kim, Jongmin Lee, Tong H. Joh, and Soo-Youl Kim. Transglutaminase 2 induces nitric oxide synthesis in BV-2 microglia. Biochem. Biophys. Res. Commun 323, 1055-1062, 2004; Jongmin Lee, Yoon-Seong Kim, Dong-Hee Choi, Moon S. Bang, Tay R. Han, Tong H. Joh, and Soo-Youl Kim. Transglutaminase 2 induces NF-κB activation via a novel pathway in BV-2 microglia. J. Biol. Chem. 279, 53725-53735, 2004; Dae-Seok Kim et al. Reversal of Drug Resistance in Breast Cancer Cells by Transglutaminase 2 Inhibition and Nuclear Factor-KB Inactivation. Cancer Res. 2006. in press Cancer Res. Nov. 15, 2006; 66(22):10936-43; Park S. S. et al., Transglutaminase 2 mediates polymer formation of 1-kappaBalpha through C-terminal glutamine cluster. J Biol. Chem. Nov. 17, 2006; 281(46):34965-72. Epub Sep. 20, 2006; Kim J M et al., A new regulatory mechanism of NF-kappaB activation by 1-kappaBbeta in cancer cells. J Mol. Biol. Dec. 26, 2008; 384(4):756-65. Epub Oct. 11, 2008; Kim D S et al., Transglutaminase 2 gene ablation protects against renal ischemic injury by blocking constant NF-κB activation. Biochem Biophys Res Commun Dec. 17, 2010; 403(3-4):479-84. Epub Nov. 19, 2010). Chemoresistance is largely attributable to the activation of NF-κB transcript material. NF-κB is known to be activated by kinases in signal transduction pathways. However, NF-κB was also found to be activated independently of kinases, thereby the effectiveness of kinase inhibitors has been redused (Tergaonkar et al., IkappaB kinase-independent IkappaBalpha degradation pathway: functional NF-kappaB activity and implications for cancer therapy. Mol Cell Biol. 2003 November; 23(22):8070-83). This mechanism has been widely regarded as a chemoresistance mechanism.

In a previous study conducted by the present inventors, it was reported that transglutaminase activates NF-κB independently of the activation of kinases (IKK, NAK), by inducing crosslinking I-κBa, and this mechanism plays an important role in chemoresistance (Jongmin Lee, et al. Transglutaminase 2 induces NF-kB activation via a novel pathway in BV-2 microglia. J. Biol. Chem. 279, 53725-53735, 2004; Dae-Seok Kim et al. Reversal of Drug Resistance in Breast Cancer Cells by Transglutaminase 2 Inhibition and Nuclear Factor-KB Inactivation. Cancer Res. Nov. 15, 2006; 66(22):10936-43s). Transglutaminase can activate NF-κB only at an intracellular level of calcium independently of the phosphorylation of kinases. In addition, this vicious cycle may be a main cause of cancer metastasis and chemo-resistance (Jongmin Lee, et al. Transglutaminase 2 induces NF-kB activation via a novel pathway in BV-2 microglia. J. Biol. Chem. 279, 53725-53735, 2004; Dae-Seok Kim et al. Reversal of Drug Resistance in Breast Cancer Cells by Transglutaminase 2 Inhibition and Nuclear Factor-KB Inactivation. Cancer Res. Nov. 15, 2006; 66(22):10936-43s; Park K S et al., TNF-alpha mediated NF-kappaB activation is constantly extended by transglutaminase 2. Front Biosci (Elite Ed). Jan. 1, 2011; 3:341-54). Therefore, many efforts have been made to find an organic small-molecule TGase 2 inhibitor and to develop it as an anti-cancer agent. As a result, it was reported that compounds having a skeleton of phenylalanine, indazol, benzopyran, or benzodioxin showed an inhibitory effect having IC50 values of several to several tens μM on TGase 2. Unfortunately, there are very few compounds reported (Lee C H, Kim S Y. NF-kB and Therapeutic Approach. Biomol & Ther 17 (3) 219-240, 2009).

Accordingly, the present inventors have studied to develop TGase 2 inhibitors. As a result, they found that 2-alkyloxy-3-phenylethynyl-4a,5-dihydropyrido[2,3-b]pyrazine derivatives show a remarkable inhibitory effect on TGase 2, completing the present invention.

### SUMMARY OF THE INVENTION

An object of the present invention is to provide a method for inhibiting TGase2 activity using 2-alkyloxy-3-phenylethynyl-4a,5-dihydropyrido[2,3-b]pyrazine derivatives.

### BRIEF DESCRIPTION OF THE DRAWINGS

FIG. 1 is a graph showing IG50 values of a Compound KROR-100721. The horizontal axis is the micro Mol concentration of KROR-100721 and the vertical axis is relative TGase 2 activity (%).

FIGS. 2A-B the above (FIG. 2A) is a graph showing the SRB test results (growth rate %) when eight renal cancer cell lines were treated by Example 1 in order to examine anti-cancer efficacy. And the following (FIG. 2B) is a graph showing the SRB test results (growth rate %) when eight renal cancer cell lines were treated by a sutent® in order to examine anti-cancer efficacy.

FIG. 3 is a graph showing comparison of inhibitory effects on transglutaminase between a compound KROR-100721 and Cisplatin, when each of them is administered into MDA-MB-231 cells.

FIG. 4 shows X-ray images of the groups treated with or without a compound KROR-100721 after subcutaneous implanting MDA-MB-231 cells to female Balb/c-nude mice.

### DETAILED DESCRIPTION OF THE PREFERRED EMBODIMENTS

The present invention provides a method for inhibiting transglutaminase 2 activity comprising contacting cells with a compound represented by the following Chemical Formula 1.

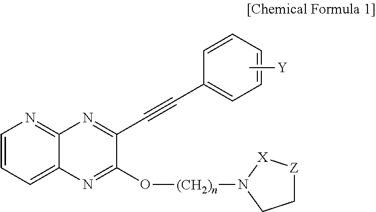

wherein Y is halogen, C₁₋₄ alkoxy, C₁₋₄ alkyl, C₁₋₄ alkylamino or C₁₋₄ haloalkyl;

### X is —CO—, —CH₂CH₂— or —CH₂—;

### Z is —NH—, —O— or —CH₂—; and

n is an integer of 1 to 3.

Preferably, X is —CO— and Z is —NH— or —CH2-.

Preferably, X is —CH2CH2- and Z is —O— or —CH2-.

Preferably, X is —CH2- and Z is —CH2-.

Preferably, Y is halogen, methoxy, methyl, dimethylamino or trifluoroalkyl.

Preferably n is 2 or 3.

Further, the compound inhibiting transglutaminase 2 activity may be any one selected from the group consisting of:
1) 3-(phenylethynyl)-2-(2-(pyrrolidin-1-yl)ethoxy)pyrido[3,2-b]pyrazine,
2) 3-(phenylethynyl)-2-(2-(piperidin-1-yl)ethoxy)pyrido[3,2-b]pyrazine,
3) 4-(2-(3-(phenylethynyl)pyrido[3,2-b]pyrazin-2-yloxy)ethyl)morpholine,
4) 3-(phenylethynyl)-2-(3-(pyrrolidin-1-yl)propoxy)pyrido[3,2-b]pyrazine,
5) 3-(phenylethynyl)-2-(3-(piperidin-1-yl)propoxy)pyrido[3,2-b]pyrazine,
6) 4-(3-(3-(phenylethynyl)pyrido[3,2-b]pyrazin-2-yloxy)propyl)morpholine,
7) 1-(3-(3-(phenylethynyl)pyrido[3,2-b]pyrazin-2-yloxy)propyl)pyrrolidin-2-one,
8) N,N-dimethyl-4-((2-(2-(pyrrolidin-1-yl)ethoxy)pyrido[3,2-b]pyrazin-3-yl)ethynyl)benzenamine,
9) N,N-dimethyl-4-((2-(2-(piperidin-1-yl)ethoxy)pyrido[3,2-b]pyrazin-3-yl)ethynyl)benzenamine,
10) 1-(2-(3-((4-(dimethylamino)phenyl)ethynyl)pyrido[3,2-b]pyrazin-2-yloxy)ethyl)pyrrolidin-2-one,
11) N,N-dimethyl-4-((2-(3-morpholinopropoxy)pyrido[3,2-b]pyrazin-3-yl)ethynyl)benzenamine,
12) 1-(3-(3-((4-(dimethylamino)phenyl)ethynyl)pyrido[3,2-b]pyrazin-2-yloxy)propyl)pyrrolidin-2-one,
13) 1-(2-(3-((4-(dimethylamino)phenyl)ethynyl)pyrido[3,2-b]pyrazin-2-yloxy)ethyl)imidazolidin-2-one,
14) 3-((4-methoxyphenyl)ethynyl)-2-(2-(pyrrolidin-1-yl)ethoxy)pyrido[3,2-b]pyrazine,
15) 3-((4-methoxyphenyl)ethynyl)-2-(2-(piperidin-1-yl)ethoxy)pyrido[3,2-b]pyrazine,
16) 1-(2-(3-((4-methoxyphenyl)ethynyl)pyrido[3,2-b]pyrazin-2-yloxy)ethyl)pyrrolidin-2-one,
17) 4-(2-(3-((4-methoxyphenyl)ethynyl)pyrido[3,2-b]pyrazin-2-yloxy)ethyl)morpholine,
18) 4-(3-(3-((4-methoxyphenyl)ethynyl)pyrido[3,2-b]pyrazin-2-yloxy)propyl)morpholine,
19) 4-(3-(3-((4-methoxyphenyl)ethynyl)pyrido[3,2-b]pyrazin-2-yloxy)propyl)morpholine,
20) 1-(2-(3-((4-methoxyphenyl)ethynyl)pyrido[3,2-b]pyrazin-2-yloxy)ethyl)imidazolidin-2-one,
21) 2-(2-(pyrrolidin-1-yl)ethoxy)-3-(p-tolylethynyl)pyrido[3,2-b]pyrazine,
22) 2-(2-(piperidin-1-yl)ethoxy)-3-(p-tolylethynyl)pyrido[3,2-b]pyrazine,
23) 1-(2-(3-(p-tolylethynyl)pyrido[3,2-b]pyrazin-2-yloxy)ethyl)pyrrolidin-2-one,
24) 4-(2-(3-(p-tolylethynyl)pyrido[3,2-b]pyrazin-2-yloxy)ethyl)morpholine,
25) 4-(3-(3-(p-tolylethynyl)pyrido[3,2-b]pyrazin-2-yloxy)propyl)morpholine,
26) 1-(3-(3-(p-tolylethynyl)pyrido[3,2-b]pyrazin-2-yloxy)propyl)pyrrolidin-2-one,
27) 1-(2-(3-(p-tolylethynyl)pyrido[3,2-b]pyrazin-2-yloxy)ethyl)imidazolidin-2-one,
28) 2-(2-(pyrrolidin-1-yl)ethoxy)-3-((4-(trifluoromethyl)phenyl)ethynyl)pyrido[3,2-b]pyrazine,
29) 2-(2-(piperidin-1-yl)ethoxy)-3-((4-(trifluoromethyl)phenyl)ethynyl)pyrido[3,2-b]pyrazine,
30) 1-(2-(3-((4-(trifluoromethyl)phenyl)ethynyl)pyrido[3,2-b]pyrazin-2-yloxy)ethyl)pyrrolidin-2-one,
31) 4-(2-(3-((4-(trifluoromethyl)phenyl)ethynyl)pyrido[3,2-b]pyrazin-2-yloxy)ethyl)morpholine,
32) 4-(3-(3-((4-(trifluoromethyl)phenyl)ethynyl)pyrido[3,2-b]pyrazin-2-yloxy)propyl)morpholine,
33) 1-(3-(3-((4-(trifluoromethyl)phenyl)ethynyl)pyrido[3,2-b]pyrazin-2-yloxy)propyl)pyrrolidin-2-one,
34) 1-(2-(3-((4-(trifluoromethyl)phenyl)ethynyl)pyrido[3,2-b]pyrazin-2-yloxy)ethyl)imidazolidin-2-one,
35) N,N-dimethyl-3-((2-(2-(pyrrolidin-1-yl)ethoxy)pyrido[3,2-b]pyrazin-3-yl)ethynyl)benzenamine,
36) N,N-dimethyl-3-((2-(2-(piperidin-1-yl)ethoxy)pyrido[3,2-b]pyrazin-3-yl)ethynyl)benzenamine,
37) 2-(2-(3-((3-(dimethylamino)phenyl)ethynyl)pyrido[3,2-b]pyrazin-2-yloxy)ethyl)cyclopentanone,
38) N,N-dimethyl-3-((2-(2-morpholinoethoxy)pyrido[3,2-b]pyrazin-3-yl)ethynyl)benzenamine,
39) N,N-dimethyl-3-((2-(3-morpholinopropoxy)pyrido[3,2-b]pyrazin-3-yl)ethynyl)benzenamine,
40) 1-(3-(3-((3-(dimethylamino)phenyl)ethynyl)pyrido[3,2-b]pyrazin-2-yloxy)propyl)pyrrolidin-2-one,
41) 1-(2-(3-((3-(dimethylamino)phenyl)ethynyl)pyrido[3,2-b]pyrazin-2-yloxy)ethyl)imidazolidin-2-one,
42) N,N-dimethyl-2-((2-(2-(pyrrolidin-1-yl)ethoxy)pyrido[3,2-b]pyrazin-3-yl)ethynyl)benzenamine,
43) N,N-dimethyl-2-((2-(2-(piperidin-1-yl)ethoxy)pyrido[3,2-b]pyrazin-3-yl)ethynyl)benzenamine,
44) 1-(2-(3-((2-(dimethylamino)phenyl)ethynyl)pyrido[3,2-b]pyrazin-2-yloxy)ethyl)pyrrolidin-2-one,
45) N,N-dimethyl-2-((2-(2-morpholinoethoxy)pyrido[3,2-b]pyrazin-3-yl)ethynyl)benzenamine,
46) N,N-dimethyl-2-((2-(3-morpholinopropoxy)pyrido[3,2-b]pyrazin-3-yl)ethynyl)benzenamine,
47) 1-(3-(3-((2-(dimethylamino)phenyl)ethynyl)pyrido[3,2-b]pyrazin-2-yloxy)propyl)pyrrolidin-2-one,
48) 1-(2-(3-((2-(dimethylamino)phenyl)ethynyl)pyrido[3,2-b]pyrazin-2-yloxy)ethyl)imidazolidin-2-one,
49) 2-(2-(pyrrolidin-1-yl)ethoxy)-3-((3-(trifluoromethyl)phenyl)ethynyl)pyrido[3,2-b]pyrazine, and
50) 2-(2-(piperidin-1-yl)ethoxy)-3-((3-(trifluoromethyl)phenyl)ethynyl)pyrido[3,2-b]pyrazine.

Further, the compound represented by Chemical Formula 1 according to the present invention may be also in a form of a solvate (e.g., hydrate).

Further, the compound represented by Chemical Formula 1 according to the present invention may have one or more chiral centers, and examples thereof may include enantiomers or diastereomers. Therefore, the present invention encompasses all such isomers or mixtures thereof.

The compound of Chemical Formula (I) according to the present invention may be prepared by a two-step process, as shown in the following Reaction Scheme 1.

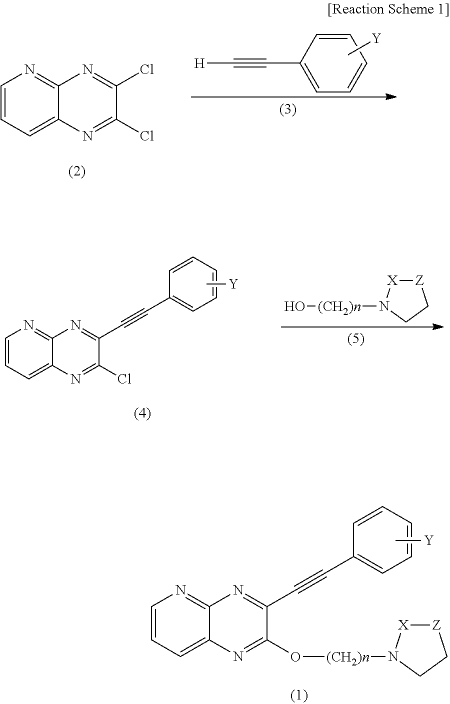

wherein X, Y, Z and n are the same as defined above.

The preparation method of the present invention according to Reaction Scheme 1 will be described in more detail as follows.

In the first step of Reaction Scheme 1, an ethynyl group donor reagent represented by Chemical Formula 3 is used in an amount of 1.0 to 1.5 equivalents, preferably 1.0 to 1.1 equivalents with reference to 2,3-dichloropyrido[2,3-b]pyrazine represented by Chemical Formula 2. Further, ‘conditions required for Sonogashira reaction’, applied to the first step of the present invention, consist of typical palladium reagents, typical ligands, typical copper reagents, typical inorganic bases or organic bases. Examples of the typical palladium reagents may include palladium (II) acetate, palladium (II) chloride, bis(benzonitrile)dichloropalladium (II), dichlorobis(triphenylphosphine)palladium (II), tris(dibenzylidineacetone)dipalladium (0), tetrakis(triphenylphosphine)palladium (0), 1,2-bis(diphenylphosphino)ethane)dichloropalladium (II), dichlorobis(tricyclohexylphosphine)palladium (II), allylpalladium(II) chloride dimer, palladium (II) trifluoracetate, dichlorobis(tri-o-tolylphosphine)palladium (II), bis(dibenzylidineacetone) palladium(0) or the like. Examples of the typical ligands may include triphenylphosphine, (2-biphenyl)dicyclohexylphosphine, 1,2-bis(diphenylphosphino)ethane, 1,4-bis(diphenylphosphino)butane, bis(diphenylphosphino)methane, 1,3-bis(diphenylphosphino)propane, 1,2-bis(dicyclohexylphosphino)ethane, bis(dicyclohexylphosphino)methane, tris(2,4,6-trimethylphenyl)phosphine, tri-t-butylphosphine, tri-o-tolylphosphine, tris(2,6-dimethoxyphenyl)phosphine, tris(2,4,6-trimethoxyphenyl)phosphine, tri-2-furylphosphine, triphenylarsine, 1,4-bis(dicyclophosphino)butane, 1,3-bis(2,4,6-trimethylphenyl)imidazolium chloride, 1,3-bis(2,6-diisopropylphenyl)imidazolium chloride, (2-biphenyl)di-t-butylphosphine or the like. Examples of the typical copper reagents may include copper iodide (I) or the like. Examples of the typical inorganic or organic bases may include sodium hydride, potassium hydride, sodium hydroxide, potassium hydroxide, sodium carbonate, potassium carbonate, cesium carbonate, sodium hydrogen carbonate, potassium hydrogen carbonate, triethylamine, diisopropylamine, diisopropylethylamine, pyridine, 2,6-lutidine, 1,8-diazabicyclo[5.4.0]-undec-7-ene (hereinafter, referred to as ‘DBU’) or the like. In the Sonogashira reaction, the typical solvents, for example, toluene, tetrahydrofuran, dioxane, dimethoxyethane, dichloromethane, acetonitrile, dimethylformamide, water or the like are used. The reaction temperature is preferably maintained in the range from 0° C. to a boiling point of the solvent, and preferably room temperature to 100° C. The reaction time is 1 to 48 hours, and preferably 2 to 24 hours.

In the second step of Reaction Scheme 1, the typical organic solvents, for example, tetrahydrofuran, dichloromethane, acetonitrile, dimethylformamide or the like, are used. The reaction temperature is preferably maintained in the range from 0° C. to a boiling point of the solvent, and preferably room temperature to 100° C. The reaction time is 1 to 48 hours, and preferably 3 to 24 hours. A compound represented by Chemical Formula 5 is used in an amount of 1.0 to 1.5 equivalents, preferably 1.0 to 1.1 equivalents with reference to a compound represented by Chemical Formula 4. Further, this reaction is preferably performed in the presence of a typical organic or inorganic base, for example, sodium hydride, potassium hydride, sodium hydroxide, potassium hydroxide, sodium carbonate, potassium carbonate, cesium carbonate, sodium hydrogen carbonate, potassium hydrogen carbonate, triethylamine, pyridine, DBU or the like.

In order to confirm the production of the compounds represented by Chemical Formula 1 according to the present invention, the resultant was separated and purified by using a multiple column chromatography (Quad3+; Biotage, USA) after the second step, and then subjected to a structural analysis with NMR and Mass spectra.

Further, 2,3-dichloropyrido[2,3-b]pyrazine represented by Chemical Formula 2, which is used as a starting material in the preparation method according to the present invention, may be prepared by a method described in Arch. Pharm. Ber. Dtsch. Pharm. Ges. 1970, 303, 44. or by a similar method thereof.

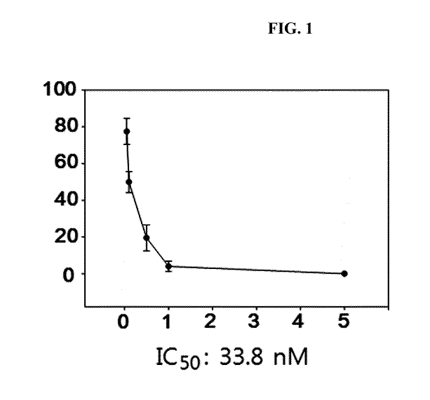

In a specific Example of the present invention, the inhibitory effect of 2-alkyloxy-3-phenylethynyl-4a,5-dihydropyrido[2,3-b]pyrazine derivative was examined by measuring transglutaminase 2 makes to combinate [1,4-C14] putrescine with succinylated casein. When 2-alkyloxy-3-phenylethynyl-4a,5-dihydropyrido[2,3-b]pyrazine derivative was added to the transglutaminase reaction combinating C14putrescine with succinylated casein in vitro, it was found that a higher concentration of 2-alkyloxy-3-phenylethynyl-4a,5-dihydropyrido[2,3-b]pyrazine derivative leads to a poorer activity of transglutaminase (FIG. 1). In particular, 2-alkyloxy-3-phenylethynyl-4a,5-dihydropyrido[2,3-b]pyrazine derivative showed the IC50 value of several nm, indicating that its effect is much better than other transglutaminase inhibitors. Consequently, it can be seen that 2-alkyloxy-3-phenylethynyl-4a,5-dihydropyrido[2,3-b]pyrazine derivative is a transglutaminase inhibitor, and 2-alkyloxy-3-phenylethynyl-4a,5-dihydropyrido[2,3-b]pyrazine derivative can reduce the increased activity of transglutaminase, even upon the overexpression of transglutaminase.

Hereinafter, the present invention will be described in more detail with reference to Examples. However, these Examples are for illustrative purposes only, and the invention is not intended to be limited by these Examples.

1H NMR spectrum was measured by Bruker Avance 300 MHz and Varian 500 MHz. The chemical shift was described in ppm concerning with TMS as the inside standard. Every example was dissolved in DMSO-d6 and CDCl3 if any other method was not described. Mass spectroscopy data was measured by Quattro Micro Moldel of Micromass Company.

### Example 1

### Synthesis of 3-phenylethynyl-2-(2-(pyrrolidin-1-yl)ethoxy)pyrido[2,3-b]pyrazine

a) Synthesis of 2-chloro-3-phenylethynylpyrido[2,3-b]pyrazine. 2,3-dichloropyrido[2,3-b]pyrazine (4.76 g, 18.5 mmol) was dissolved in dimethyl sulfoxide (2 mL) at room temperature, and then phenylacetylene (2.3 mL, 21.3 mmol), triethylamine (18.0 mL, 129.6 mmol), palladium(II) acetate (290 mg, 1.3 mmol), copper iodide(I) (437 mg, 1.7 mmol), and triphenylphosphine (388 mg, 2.0 mmol) were added thereto. The mixture was stirred at 60° C. for 2 hours. The reaction mixture was cooled at room temperature, and concentrated under reduced pressure. The obtained concentrate was extracted using water and ethyl acetate. An organic layer was dried over anhydrous magnesium sulfate, and then the resultant was purified by silica gel column chromatography (hexane/dichloromethane, 2/3, v/v) to obtain the title compound (4.6 g, 83%). 1H NMR (500 MHz, CDCl3) δ 7.46 (m, 6H), 7.73 (m, 6H), 8.34 (d, J=8.3 Hz, 2H), 9.19 (dd, J=2.31, 1.8 Hz, 2H); MS (ESI) m/z 266 ([M+H]+).

b) Synthesis of 3-phenylethynyl-2-(2-(pyrrolidin-1-yl)ethoxy)pyrido[2,3-b]pyrazine. 2-Hydroxypyrrolidine (11.4 μL, 0.097 mmol) and 60% sodium hydride (6 mg, 0.15 mmol) were stirred in tetrahydrofuran (2 mL) at room temperature for 20 minutes, and then 2-chloro-3-phenylethynylpyrido[2,3-b]pyrazine (30 mg, 0.097 mmol) was added thereto. The mixture was stirred at the same temperature for 10 hours, and the reactant was concentrated under reduced pressure. The resultant was purified by silica gel column chromatography (dichloromethane/ethanol, 9/1, v/v) to obtain the title compound (9 mg, 27%). 1H NMR (500 MHz, CDCl3) δ 1.79-1.84 (m, 4H), 2.76-2.79 (m, 4H), 3.06 (t, J=5.7 Hz, 2H), 4.73 (t, J=5.7 Hz, 2H), 7.40-7.43 (m, 3H), 7.57 (dd, J=8.3, 4.3 Hz, 1H), 7.66 (dd, J=7.7, 1.4 Hz, 2H), 8.14 (dd, J=8.3, 1.8 Hz, 1H), 8.95 (dd, J=4.3, 1.8 Hz, 1H); MS (ESI) m/z 345 ([M+H]+).

### Example 2

Synthesis of 3-phenylethynyl-2-(2-(piperidin-1-yl)ethoxy)pyrido[2,3-b]pyrazine. The study was performed in the same manner as in b) of Example 1 to obtain the title compound (11.5 mg, 33%). 1H NMR (300 MHz, CDCl3) δ 1.45 (m, 2H), 1.60 (m, 4H), 2.63 (m, 4H), 2.93 (t, J=5.7 Hz, 2H), 4.71 (t, J=5.7 Hz, 2H), 7.40-7.43 (m, 3H), 7.57 (dd, J=8.3, 4.3 Hz, 1H), 7.66 (dd, J=7.7, 1.4 Hz, 2H), 8.14 (dd, J=8.3, 1.8 Hz, 1H), 8.95 (dd, J=4.3, 1.8 Hz, 1H); MS (ESI) m/z 359 ([M+H]+).

### Example 3

Synthesis of 4-(2-(3-(phenylethynyl)pyrido[3,2-b]pyrazin-2-yloxy)ethyl)morpholine. The study was performed in the same manner as in b) of Example 1 to obtain the title compound (11.5 mg, 31%). 1H NMR (300 MHz, DMSO-d6) δ 2.58 (t, J=9.6 Hz, 4H), 2.86 (t, J=5.3 Hz, 2H), 3.53 (t, J=4.5 Hz, 4H), 4.65 (t, J=5.4 Hz, 2H), 7.52-7.57 (m, 3H), 7.68-7.71 (m, 2H), 7.78 (dd, J=8.3 Hz, 4.6 Hz, 1H), 8.27 (dd, J=8.5 Hz, 2.0 Hz, 1H), 8.96 (dd, J=4.3 Hz, 1.9 Hz, 1H); MS (ESI) m/z 361 ([M+H]⁺).

### Example 4

Synthesis of 3-(phenylethynyl)-2-(3-(pyrrolidin-1-yl)propoxy)pyrido[3,2-b]pyrazine. The study was performed in the same manner as in b) of Example 1 to obtain the title compound (8.7 mg, 25%). 1H NMR (300 MHz, CDCl3) δ 1.86 (m, 4H), 2.23 (quintet, J=6.4 Hz, 2H), 2.69 (m, 4H), 2.85 (t, J=6.4 Hz, 2H), 4.64 (t, J=6.4 Hz, 2H), 7.35-7.49 (m, 3H), 7.58 (dd, J=8.3, 4.3 Hz, 1H), 7.68 (dd, J=7.7, 1.4 Hz, 2H), 8.15 (dd, J=8.3, 1.8 Hz, 1H), 8.95 (dd, J=4.3, 1.8 Hz, 1H)

### Example 5

Synthesis of 3-(phenylethynyl)-2-(3-(piperidin-1-yl)propoxy)pyrido[3,2-b]pyrazine. The study was performed in the same manner as in b) of Example 1 to obtain the title compound (11.5 mg, 31%). MS (ESI) m/z 372.20 ([M+H]+).

### Example 6

Synthesis of 4-(3-(3-(phenylethynyl)pyrido[3,2-b]pyrazin-2-yloxy)propyl)morpholine. The study was performed in the same manner as in b) of Example 1 to obtain the title compound (11.5 mg, 31%). MS (ESI) m/z 374.17 ([M+H]+).

### Example 7

Synthesis of 1-(3-(3-(phenylethynyl)pyrido[3,2-b]pyrazin-2-yloxy)propyl)pyrrolidin-2-one. The study was performed in the same manner as in b) of Example 1 to obtain the title compound (11.5 mg, 31%). MS (ESI) m/z 372.16 ([M+H]+).

### Example 8

Synthesis of N,N-dimethyl-4-((2-(2-(pyrrolidin-1-yl)ethoxy)pyrido[3,2-b]pyrazin-3-yl)ethynyl)benzenamine. The study was performed in the same manner as in Example 1 to obtain the title compound (11.2 mg, 30%).

1H NMR (300 MHz, CDCl3) δ 1.80-1.85 (m, 4H), 2.75-2.80 (m, 4H), 3.05 (s, 6H), 3.08 (t, J=6.5 Hz, 2H), 4.73 (t, J=6.5 Hz, 2H), 6.69 (d, J=8.9 Hz, 2H), 7.53 (dd, J=8.3, 4.3 Hz, 1H), 7.55 (d, J=8.9 Hz, 2H), 8.12 (dd, J=8.3, 1.8 Hz, 1H), 8.93 (dd, J=4.3, 1.8 Hz, 1H); MS (ESI) m/z 388 ([M+H]+).

### Example 9

Synthesis of N,N-dimethyl-4-((2-(2-(piperidin-1-yl)ethoxy)pyrido[3,2-b]pyrazin-3-yl)ethynyl)benzenamine. The study was performed in the same manner as in Example 1 to obtain the title compound (13.6 mg, 35%). 1H NMR (300 MHz, CDCl3) δ 1.40-1.50 (m, 2H), 1.60-1.70 (m, 4H), 1.65-1.75 (m, 4H), 2.97 (t, J=6.5 Hz, 2H), 3.05 (s, 6H), 4.72 (t, J=6.5 Hz, 2H), 6.69 (d, J=8.9 Hz, 2H), 7.53 (dd, J=8.3, 4.3 Hz, 1H), 7.56 (d, J=8.9 Hz, 2H), 8.12 (dd, J=8.3, 1.8 Hz, 1H), 8.93 (dd, J=4.3, 1.8 Hz, 1H); MS (ESI) m/z 403 ([M+H]+).

### Example 10

Synthesis of 1-(2-(3-((4-(dimethylamino)phenyl)ethynyl)pyrido[3,2-b]pyrazin-2-yloxy)ethyl)pyrrolidin-2-one. The study was performed in the same manner as in Example 1 to obtain the title compound (11.5 mg, 31%). 1H NMR (300 MHz, CDCl3) δ 2.04 (quintet, J=7.7 Hz, 2H), 2.42 (t, J=7.7 Hz, 2H), 3.77 (t, J=7.7 Hz, 2H), 3.86 (t, J=5.1 Hz, 2H), 4.70 (t, J=5.1 Hz, 2H), 6.69 (d, J=8.9 Hz, 2H), 7.55 (d, J=8.9 Hz, 2H), 7.56 (dd, J=8.3 and 4.3 Hz, 1H), 8.13 (dd, J=8.3 and 1.8 Hz, 1H), 8.95 (dd, J=4.3 and 1.8 Hz, 1H.

### Example 11

Synthesis of N,N-dimethyl-4-((2-(3-morpholinopropoxy)pyrido[3,2-b]pyrazin-3-yl)ethynyl)benzenamine. The study was performed in the same manner as in Example 1 to obtain the title compound (11.5 mg, 31%). 1H NMR (300 MHz, CDCl3) δ 2.47-2.73 (m, 2H), 2.47-2.73 (m, 6H), 3.04 (s, 6H), 3.67-3.78 (m, 4H), 4.62 (t, J=6.5 Hz, 2H), 6.68 (d, J=8.8 Hz, 2H), 7.48-7.58 (m, 3H), 8.10 (dd, J=8.0 Hz, 1.9 Hz, 1H), 8.94 (dd, J=4.2 Hz, 1.9 Hz, 1H)

### Example 12

Synthesis of 1-(3-(3-((4-(dimethylamino)phenyl)ethynyl)pyrido[3,2-b]pyrazin-2-yloxy)propyl)pyrrolidin-2-one. The study was performed in the same manner as in Example 1 to obtain the title compound (11.5 mg, 31%). 1H NMR (300 MHz, CDCl3) δ 1.97-2.07 (m, 2H), 2.14-2.24 (m, 2H), 2.39 (t, J=7.6 Hz, 2H), 3.05 (s, 6H), 3.49 (t, J=7.2 Hz, 2H), 3.59 (t, J=6.9 Hz, 2H), 4.60 (t, J=6.1 Hz, 2H), 6.69 (d, J=9.2 HZ, 2H), 7.50-7.58 (m, 3H), 8.11 (dd, J=8.4 Hz, J=1.9 Hz, 1H), 8.92 (dd, J=4.2 Hz, J=1.9 Hz, 1H)

### Example 13

Synthesis of 1-(2-(3-((4-(dimethylamino)phenyl)ethynyl)pyrido[3,2-b]pyrazin-2-yloxy)ethyl)imidazolidin-2-one. The study was performed in the same manner as in Example 1 to obtain the title compound (11.5 mg, 31%). MS (ESI) m/z 402.18 ([M+H]+).

### Example 14

Synthesis of 3-(4-methoxyphenylethynyl)-2-(2-pyrrolidin-1-ylethoxy)pyrido[3,2-b]pyrazine. The study was performed in the same manner as in Example 1 to obtain the title compound (13.4 mg, 37%). 1H NMR (300 MHz, CDCl3) δ 1.88 (br. s, 4H), 2.83 (br. s, 4H), 3.21 (t, J=5.6 Hz, 2H), 3.87 (s, 3H), 4.80 (t, J=5.6 Hz, 2H), 6.91-6.94 (m, 2H), 7.56-7.62 (m, 3H), 8.14 (dd, J=1.5, 8.0 Hz, 1H), 8.96 (dd, J=1.5, 4.1 Hz, 1H); MS (ESI) m/z 375 ([M+H]+).

### Example 15

Synthesis of 3-(4-methoxyphenylethynyl)-2-(2-piperidin-1-ylethoxy)pyrido[3,2-b]pyrazine. The study was performed in the same manner as in Example 1 to obtain the title compound (12 mg, 32%). 1H NMR (300 MHz, CDCl3) δ 1.47 (br. s, 2H), 1.63-1.67 (m, 4H), 2.72 (br. s, 4H), 3.01 (t, J=5.7 Hz, 2H), 3.86 (s, 3H), 4.75 (t, J=5.7 Hz, 2H), 6.93 (dd, J=1.9, 7.0 Hz, 2H), 7.56 (q, J=4.3 Hz, 1H), 7.61 (dd, J=1.9, 6.9 Hz, 2H), 8.13 (dd, J=1.8, 8.3 Hz, 1H), 8.95 (dd, J=1.8, 4.3 Hz, 1H); MS (ESI) m/z 389 ([M+H]+).

### Example 16

Synthesis of 1-(2-(3-((4-methoxyphenyl)ethynyl)pyrido[3,2-b]pyrazin-2-yloxy)ethyl)pyrrolidin-2-one. The study was performed in the same manner as in Example 1 to obtain the title compound (11.5 mg, 31%). MS (ESI) m/z 388.15 ([M+H]+).

### Example 17

Synthesis of 4-(2-(3-((4-methoxyphenyl)ethynyl)pyrido[3,2-b]pyrazin-2-yloxy)ethyl)morpholine. The study was performed in the same manner as in Example 1 to obtain the title compound (11.5 mg, 31%). 1H NMR (300 MHz, CDCl3) δ 2.72 (br. s, 4H), 2.96 (t, J=5.6 Hz, 2H), 3.73 (t, J=4.7 Hz, 4H), 3.86 (s, 3H), 4.72 (t, J=5.6 Hz, 2H), 6.92-6.94 (m, 2H), 7.56-7.61 (m, 3H), 8.13 (dd, J=1.8, 8.3 Hz, 1H), 8.95 (dd, J=1.7, 4.2 Hz, 1H).

### Example 18

Synthesis of 4-(3-(3-((4-methoxyphenyl)ethynyl)pyrido[3,2-b]pyrazin-2-yloxy)propyl)morpholine. The study was performed in the same manner as in Example 1 to obtain the title compound (11.5 mg, 31%). 1H NMR (300 MHz, CDCl3) δ 2.12-2.16 (m, 2H), 2.52 (br. s, 4H), 2.65 (t, 7.2, 2H), 3.73 (t, 4.5, 4H), 3.88 (s, 3H), 4.62 (t, 6.4, 2H), 6.93 (d, 7.0, 2H), 7.56 (q, 4.2, 1H), 7.62 (dd, 1.9, 7.0, 2H), 8.13 (dd, 1.7, 8.2, 1H), 8.94 (dd, 1.8, 4.2, 1H).

### Example 19

Synthesis of 4-(3-(3-((4-methoxyphenyl)ethynyl)pyrido[3,2-b]pyrazin-2-yloxy)propyl)morpholine. The study was performed in the same manner as in Example 1 to obtain the title compound (11.5 mg, 31%). MS (ESI) m/z 266 ([M+H]+).

### Example 20

Synthesis of 1-(2-(3-((4-methoxyphenyl)ethynyl)pyrido[3,2-b]pyrazin-2-yloxy)ethyl)imidazolidin-2-one. The study was performed in the same manner as in Example 1 to obtain the title compound (11.5 mg, 31%). MS (ESI) m/z 389.15 ([M+H]+).

### Example 21

Synthesis of 2-(2-(pyrrolidin-1-yl)ethoxy)-3-(p-tolylethynyl)pyrido[3,2-b]pyrazine. The study was performed in the same manner as in Example 1 to obtain the title compound (10 mg, 30%). 1H NMR (300 MHz, CDCl3) δ 1.85 (s, 4H), 2.41 (s, 3H), 2.83 (s, 4H), 3.10 (t, J=5.7 Hz, 2H), 4.75 (t, J=5.7, 2H), 7.21 (d, J=8.0 Hz, 2H), 7.55-7.58 (m, 3H), 8.14 (dd, J=1.8, 8.3 Hz, 1H), 8.95 (dd, J=1.8. 4.2 Hz, 1H); MS (ESI) m/z 359 ([M+H]+).

### Example 22

Synthesis of 2-(2-(piperidin-1-yl)ethoxy)-3-(p-tolylethynyl)pyrido[3,2-b]pyrazine. The study was performed in the same manner as in b) of Example 1 to obtain the title compound (11.5 mg, 31%). MS (ESI) m/z 372.20 ([M+H]+).

### Example 23

Synthesis of 1-(2-(3-(p-tolylethynyl)pyrido[3,2-b]pyrazin-2-yloxy)ethyl)pyrrolidin-2-one. The study was performed in the same manner as in Example 1 to obtain the title compound (11.5 mg, 31%). 1H NMR (300 MHz, CDCl3) δ 2.00 (m, 2H), 2.38 (t, J=7.1 Hz, 2H), 2.41 (s, 3H), 3.72 (t, J=7.1 Hz, 2H), 3.84 (t, J=5.1 Hz, 2H), 4.70 (t, J=5.1 Hz, 2H), 7.23 (d, J=8.1 Hz, 2H), 7.55 (d, J=8.1 Hz, 2H), 7.59 (dd, J=8.3 and 4.3 Hz, 1H), 8.14 (dd, J=8.3 and 1.8 Hz, 1H), 8.97 (dd, J=4.3 and 1.8 Hz, 1H

### Example 24

Synthesis of 4-(2-(3-(p-tolylethynyl)pyrido[3,2-b]pyrazin-2-yloxy)ethyl)morpholine. The study was performed in the same manner as in Example 1 to obtain the title compound (11.5 mg, 31%). 1H NMR (300 MHz, CDCl3) δ 2.42 (s, 3H), 2.72 (s, 4H), 2.95 (t, J=5.6 Hz, 2H), 3.72 (t, J=4.6 Hz, 4H), 4.72 (t, J=5.6 Hz, 2H), 7.22 (d, J=8.2 Hz, 2H), 7.54-7.59 (m, 3H), 8.14 (dd, J=1.7, 8.3 Hz, 1H), 8.95 (dd, J=1.8. 4.2 Hz, 1H)

### Example 25

Synthesis of 4-(3-(3-(p-tolylethynyl)pyrido[3,2-b]pyrazin-2-yloxy)propyl)morpholine. The study was performed in the same manner as in Example 1 to obtain the title compound (11.5 mg, 31%). 1H NMR (300 MHz, CDCl3) δ 2.28 (br. s, 2H), 2.41 (s, 3H), 2.64 (br. s, 4H), 2.77 (br. s, 2H), 3.81 (br. s, 4H), 4.64 (t, J=6.2 Hz, 2H), 7.22 (d, J=8.1 Hz, 2H), 7.55-7.59 (m, 3H), 8.13 (dd, J=1.7, 8.3 Hz, 1H), 8.95 (dd, J=1.8. 4.2 Hz, 1H).

### Example 26

Synthesis of 1-(3-(3-(p-tolylethynyl)pyrido[3,2-b]pyrazin-2-yloxy)propyl)pyrrolidin-2-one. The study was performed in the same manner as in Example 1 to obtain the title compound (11.5 mg, 31%). MS (ESI) m/z 386.17 ([M+H]+).

### Example 27

Synthesis of 1-(2-(3-(p-tolylethynyl)pyrido[3,2-b]pyrazin-2-yloxy)ethyl)imidazolidin-2-one. The study was performed in the same manner as in Example 1 to obtain the title compound (11.5 mg, 31%). MS (ESI) m/z 373.15 ([M+H]+).

### Example 28

Synthesis of 2-(2-(pyrrolidin-1-yl)ethoxy)-3-((4-(trifluoromethyl)phenyl)ethynyl)pyrido[3,2-b]pyrazine. The study was performed in the same manner as in b) of Example 1 to obtain the title compound (11.5 mg, 31%). MS (ESI) m/z 412.15 ([M+H]+).

### Example 29

Synthesis of 2-(2-(piperidin-1-yl)ethoxy)-3-((4-(trifluoromethyl)phenyl)ethynyl)pyrido[3,2-b]pyrazine. The study was performed in the same manner as in Example 1 to obtain the title compound (11.5 mg, 31%). MS (ESI) m/z 426.17 ([M+H]+).

### Example 30

Synthesis of 1-(2-(3-((4-(trifluoromethyl)phenyl)ethynyl)pyrido[3,2-b]pyrazin-2-yloxy)ethyl)pyrrolidin-2-one. The study was performed in the same manner as in Example 1 to obtain the title compound (11.5 mg, 31%). MS (ESI) m/z 426.13 ([M+H]+).

### Example 31

Synthesis of 4-(2-(3-((4-(trifluoromethyl)phenyl)ethynyl)pyrido[3,2-b]pyrazin-2-yloxy)ethyl)morpholine. The study was performed in the same manner as in Example 1 to obtain the title compound (11.5 mg, 31%). MS (ESI) m/z 428.15 ([M+H]+).

### Example 32

Synthesis of 4-(3-(3-((4-(trifluoromethyl)phenyl)ethynyl)pyrido[3,2-b]pyrazin-2-yloxy)propyl)morpholine. The study was performed in the same manner as in Example 1 to obtain the title compound (11.5 mg, 31%). MS (ESI) m/z 442.16 ([M+H]+).

### Example 33

Synthesis of 1-(3-(3-((4-(trifluoromethyl)phenyl)ethynyl)pyrido[3,2-b]pyrazin-2-yloxy)propyl)pyrrolidin-2-one. The study was performed in the same manner as in Example 1 to obtain the title compound (11.5 mg, 31%). MS (ESI) m/z 440.15 ([M+H]+).

### Example 34

Synthesis of 1-(2-(3-((4-(trifluoromethyl)phenyl)ethynyl)pyrido[3,2-b]pyrazin-2-yloxy)ethyl)imidazolidin-2-one. The study was performed in the same manner as in Example 1 to obtain the title compound (11.5 mg, 31%). MS (ESI) m/z 427.13 ([M+H]+).

### Example 35

Synthesis of N,N-dimethyl-3-((2-(2-(pyrrolidin-1-yl)ethoxy)pyrido[3,2-b]pyrazin-3-yl)ethynyl)benzenamine. The study was performed in the same manner as in Example 1 to obtain the title compound (11.5 mg, 31%). MS (ESI) m/z 387.21 ([M+H]+).

### Example 36

Synthesis of N,N-dimethyl-3-((2-(2-(piperidin-1-yl)ethoxy)pyrido[3,2-b]pyrazin-3-yl)ethynyl)benzenamine. The study was performed in the same manner as in Example 1 to obtain the title compound (11.5 mg, 31%). MS (ESI) m/z 401.22 ([M+H]+).

### Example 37

Synthesis of 2-(2-(3-((3-(dimethylamino)phenyl)ethynyl)pyrido[3,2-b]pyrazin-2-yloxy)ethyl)cyclopentanone. The study was performed in the same manner as in Example 1 to obtain the title compound (11.5 mg, 31%). MS (ESI) m/z 400.19 ([M+H]+).

### Example 38

Synthesis of N,N-dimethyl-3-((2-(2-morpholinoethoxy)pyrido[3,2-b]pyrazin-3-yl)ethynyl)benzenamine. The study was performed in the same manner as in Example 1 to obtain the title compound (11.5 mg, 31%). MS (ESI) m/z 403.20 ([M+H]+).

### Example 39

Synthesis of N,N-dimethyl-3-((2-(3-morpholinopropoxy)pyrido[3,2-b]pyrazin-3-yl)ethynyl)benzenamine. The study was performed in the same manner as in Example 1 to obtain the title compound (11.5 mg, 31%). MS (ESI) m/z 417.22 ([M+H]+).

### Example 40

Synthesis of 1-(3-(3-((3-(dimethylamino)phenyl)ethynyl)pyrido[3,2-b]pyrazin-2-yloxy)propyl)pyrrolidin-2-one. The study was performed in the same manner as in Example 1 to obtain the title compound (11.5 mg, 31%). MS (ESI) m/z 415.20 ([M+H]+).

### Example 41

Synthesis of 1-(2-(3-((3-(dimethylamino)phenyl)ethynyl)pyrido[3,2-b]pyrazin-2-yloxy)ethyl)imidazolidin-2-one. The study was performed in the same manner as in Example 1 to obtain the title compound (11.5 mg, 31%). MS (ESI) m/z 402.18 ([M+H]+).

### Example 42

Synthesis of N,N-dimethyl-2-((2-(2-(pyrrolidin-1-yl)ethoxy)pyrido[3,2-b]pyrazin-3-yl)ethynyl)benzenamine. The study was performed in the same manner as in Example 1 to obtain the title compound (13 mg, 35%). ¹H NMR (300 MHz, CDCl3) δ 1.87 (s, 4H), 2.98 (s, 4H), 3.11 (s, 6H), 3.14 (t, J=5.9 Hz, 2H), 4.77 (t, J=5.9 Hz, 2H), 6.87-6.92 (m, 2H), 7.31 (t, J=7.4 Hz, 1H), 7.55-7.60 (m, 2H), 8.14 (dd, J=1.8, 8.2 Hz, 1H), 8.94 (dd, J=1.8, 4.2 Hz, 1H); MS (ESI) m/z 388 ([M+H]+).

### Example 43

Synthesis of N,N-dimethyl-2-((2-(2-(piperidin-1-yl)ethoxy)pyrido[3,2-b]pyrazin-3-yl)ethynyl)benzenamine. The study was performed in the same manner as in Example 1 to obtain the title compound (11.5 mg, 31%). ¹H NMR (300 MHz, CDCl3) δ 1.46 (br. s, 2H), 1.65 (p, J=5.7 Hz, 4H), 2.71 (br. s, 4H), 3.02 (t, J=5.9 Hz, 2H), 3.12 (s, 6H), 4.77 (t, J=5.9 Hz, 2H), 6.88-6.92 (m, 2H), 7.32 (t, J=7.4 Hz, 1H), 7.55-7.61 (m, 2H), 8.12 (dd, J=1.8, 8.2 Hz, 1H), 8.94 (dd, J=1.8, 4.2 Hz, 1H).

### Example 44

Synthesis of 1-(2-(3-((2-(dimethylamino)phenyl)ethynyl)pyrido[3,2-b]pyrazin-2-yloxy)ethyl)pyrrolidin-2-one. The study was performed in the same manner as in Example 1 to obtain the title compound (11.5 mg, 31%).

1H NMR (300 MHz, CDCl3) δ 1.94-2.00 (m, 2H), 2.38 (t, J=7.0 Hz, 2H), 3.14 (s, 6H), 3.66 (t, J=7.0 Hz, 2H), 3.82 (t, J=5.9 Hz, 2H), 4.72 (t, J=5.9 Hz, 2H), 5.30 (s, 2H), 6.87-6.93 (m, 2H), 7.34 (t, J=7.4 Hz, 1H), 7.57-7.59 (m, 2H), 8.15 (dd, J=1.8, 8.2 Hz, 1H), 8.96 (dd, J=1.8, 4.2 Hz, 1H).

### Example 45

Synthesis of N,N-dimethyl-2-((2-(2-morpholinoethoxy)pyrido[3,2-b]pyrazin-3-yl)ethynyl)benzenamine. The study was performed in the same manner as in Example 1 to obtain the title compound (11.5 mg, 31%). 1H NMR (300 MHz, CDCl3) δ 2.62 (br. s, 4H), 2.95 (t, J=5.9 Hz, 2H), 3.12 (s, 6H), 3.71 (t, J=4.5 Hz, 4H), 4.72 (t, J=5.9 Hz, 2H), 4.72 (t, J=5.9 Hz, 2H), 6.88-6.92 (m, 2H), 7.32 (t, J=7.4 Hz, 1H), 7.55-7.60 (m, 2H), 8.13 (dd, J=1.8, 8.2 Hz, 1H), 8.95 (dd, J=1.8, 4.2 Hz, 1H).

### Example 46

Synthesis of N,N-dimethyl-2-((2-(3-morpholinopropoxy)pyrido[3,2-b]pyrazin-3-yl)ethynyl)benzenamine. The study was performed in the same manner as in Example 1 to obtain the title compound (11.5 mg, 31%). 1H NMR (300 MHz, CDCl3) δ 2.09-2.15 (m, 2H), 2.49 (br. s, 4H), 2.61 (t, J=7.1 Hz, 2H), 3.12 (s, 6H), 3.72 (t, J=4.5 Hz, 4H), 4.62 (t, J=6.5 Hz, 2H), 6.88-6.92 (m, 2H), 7.31 (t, J=7.4 Hz, 1H), 7.56 (q, J=4.2 Hz, 1H), 7.61 (dd, J=1.6, 7.6 Hz, 1H), 8.11 (dd, J=1.7, 8.2 Hz, 1H), 8.94 (dd, J=1.8, 4.2 Hz, 1H)

### Example 47

Synthesis of 1-(3-(3-((2-(dimethylamino)phenyl)ethynyl)pyrido[3,2-b]pyrazin-2-yloxy)propyl)pyrrolidin-2-one. The study was performed in the same manner as in Example 1 to obtain the title compound (11.5 mg, 31%). MS (ESI) m/z 415.20 ([M+H]+).

### Example 48

Synthesis of 1-(2-(3-((2-(dimethylamino)phenyl)ethynyl)pyrido[3,2-b]pyrazin-2-yloxy)ethyl)imidazolidin-2-one. The study was performed in the same manner as in Example 1 to obtain the title compound (11.5 mg, 31%). MS (ESI) m/z 402.18 ([M+H]+).

### Example 49

Synthesis of 2-(2-(piperidin-1-yl)ethoxy)-3-((3-(trifluoromethyl)phenyl)ethynyl)pyrido[3,2-b]pyrazine. The study was performed in the same manner as in Example 1 to obtain the title compound (11.5 mg, 31%). MS (ESI) m/z 412.15 ([M+H]+).

### Example 50

Synthesis of 2-(2-(piperidin-1-yl)ethoxy)-3-((3-(trifluoromethyl)phenyl)ethynyl)pyrido[3,2-b]pyrazine. The study was performed in the same manner as in Example 1 to obtain the title compound (11.5 mg, 31%). MS (ESI) m/z 426.17 ([M+H]+).

### Studyal Example 1

In vitro Assay for Inhibitory effect of 2-alkyloxy-3-phenylethynyl-4a,5-dihydropyrido[2,3-b]pyrazine derivative on TGase 2. In order to determine whether the compounds prepared in the Examples specifically inhibit TGase 2, the following study was performed in vitro.

Method. The transglutaminase make to combinate [1,4,-14C] putrescine with succinylated casein was measured to examine whether the compounds of the present invention inhibits the reaction by competition with putrescine. Succinylated casein was purchased from Calbiochem (Cat. No. 573464), and 1 g of the powder was dissolved in 50 ml of a reaction buffer solution containing 5 mM DTT (0.1 M Tris-acetate (pH 8.0), 10 mM CaCl2, 0.15 M NaCl, 1.0 mM EDTA). This solution was stored in a deep freezer for further use. [1,4-14C] Putrescine dihydrochloride was purchased from GE Healthcare (Cat. No. CFA301), and the stock solution was diluted with distilled water to yield a radiological dose of 5 μCi/ml. Transglutaminase 2 was purchased from Sigma-Aldrich (Cat. No. T5398), and diluted with distilled water to yield a final concentration of 1 unit/ml. 2-Alkyloxy-3-phenylethynyl-4a,5-dihydropyrido[2,3-b]pyrazine derivative was dissolved at a concentration of 1 mM to prepare its stock solution, and the stock solution was diluted with DMSO to prepare solutions having various concentrations.

450 μl of succinylated casein solution and 50 μl of [1,4-14C] putrescine dihydrochloride solution were mixed together to prepare a substrate solution. 96 μl of the reaction buffer, 3 μl of the stock solution of the compounds of the present invention, and 1 μl of the stock solution of transglutaminase were mixed together to prepare each sample, followed by incubation at 37° C. for 10 min. 500 μl of the substrate solution and 100 μl of sample solution were mixed well, and the mixture was incubated at 37° C. for 2 hrs and then the reaction terminated by adding 4.5 ml of cold (4° C.) 7.5% TCA. The final solution was stored at 4° C. for 1 hr. The TCA-protein precipitates were filtered through a GF/glass fiber filter, washed with 25 ml of cold 5% TCA, and dried. Radioactivity of cross-linked protein was measured using a β-counter (Beckman Coulter), and compensated by the activity of DMSO-control group as a standard. The activity of transglutaminase was represented by the measured values.

Result. The results are shown in Table 1. The results mean inhibition (%) of cell growth at 10 μM by 2-alkyloxy-3-phenylethynyl-4a,5-dihydropyrido[2,3-b]pyrazine compounds against transglutaminase cell lines.

TABLE 1
Inhibition (%) of cell growth at 10 μM by 2-alkyloxy-
3-phenylethynyl-4a,5-dihydropyrido[2,3-b]pyrazine
compounds against transglutaminase cell lines

| Example No. | Inhibition (%) |
|---|---|
| 1 | 91 |
| 2 | 83 |
| 3 | 47 |
| 4 | 59 |
| 5 | 54 |
| 6 | 32 |
| 7 | 43 |
| 8 | 68 |
| 9 | 57 |
| 14 | 64 |
| 15 | 51 |
| 21 | 52 |
| 42 | 63 |
| 43 | 54 |

Further, IC50 value of Example 1 was measured and the results are shown in FIG. 1. As shown in FIG. 1, Example 1 showed very excellent IC50 value of 33.8 nM, indicating that Example 1 has more excellent inhibitory effect than any other TGase 2 inhibitors. Therefore, it can be seen that the compounds of the present invention inhibit the activity of transglutaminase in a concentration-dependent manner.

### Studyal Example 2

Test of Anti-cancer efficacy in the Cancer cell line. In order to examine anti-cancer efficacy of 2-alkyloxy-3-phenylethynyl-4a,5-dihydropyrido[2,3-b]pyrazine derivatives, Eight renal cancer cell lines were treated to perform an SRB test. In order to compare the anti-cancer efficacy, sutent® (Pfizer) was used for comparison.

1) Method (SRB Test). The cancer cell lines were cultured in an RPMI 1640 medium containing 5% fetal bovine serum and 2 mM L-glutamine. The cancer cells were inoculated into a 96 well microtiter plate at a density of 5,000 or 40,000 cells/well. The cells were incubated at 37° C., 5% CO2, 95% air and 100% humidity for 24 hours prior to drug treatment.

After 24 hours, the cells were fixed with TCA at the time of drug addition (Tz). The drug was treated by 10-fold or log scale serial dilutions, and the efficacy was examined at a concentration of 5 point or more. After drug treatment, the cells were incubated for 48 hours. After incubation, the cells were fixed by the addition of TCA to be a final concentration of 10% after incubating the cells 1 hour at 4° C. Thereafter, the cells were washed five times and dried. Then, sulforhodamine B (SRB) solution (100 μl) was added, and incubated for 10 minutes at room temperature. The staining solution was removed, and unbound dye was removed by washing five times. Bound stain was subsequently solubilized with trizma base, and the absorbance was read at 515 nm.

Growth inhibitory efficacy was measured by using the seven absorbance measurements. [time zero, (Tz), control growth, (C), and test growth in the presence of drug at the five concentration levels (Ti)], the percentage growth was calculated at each of the drug concentrations levels.

[(Ti−Tz)/(C−Tz)]×100 for concentrations for which Ti>/=Tz

[(Ti−Tz)/Tz]×100 for concentrations for which Ti<Tz.

Growth inhibition of 50% (GI50) was calculated from [(Ti−Tz)/(C−Tz)]×100=50, which is the drug concentration resulting in a 50% reduction in the net protein increase in control cells.

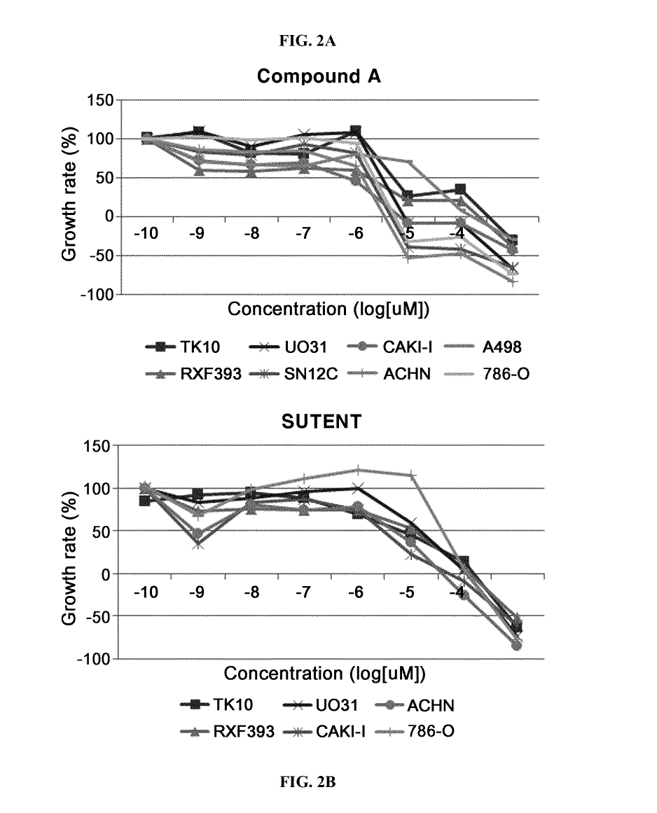

2) Result. The anti-cancer efficacy of Example 1 was found to be better than that of SUTENT® in the renal cancer cell line (FIG. 2). The IG50 values are summarized in the following Table.

TABLE 2

| RCC | IG50 (×E−7) |
|---|---|
| 786-O | 2.25 |
| ACHN | 1.37 |
| A498 | 56.14 |
| CAKI-I | 0.71 |
| RXF393 | 1.79 |
| SN12C | 1.86 |
| TK10 | 5.29 |
| US31 | 54.88 |

* Average GI50 over all cell lines is 4.30E−7

### Studyal Example 3

Evaluation of Anti-cancer efficacy of TGase 2 inhibitor on MDA231 breast cancer cell line by animal study. Chemoresistant cancer cell-injected animal models were treated with the present small-molecule selected as a TGase 2 inhibitor and an anti-cancer agent cisplatin, and the results are compared as follows.

1) Method. A. Cell culture. MDA-MB-231 cells were cultured in a DMEM medium containing 10% FBS.

B. Animal model. 8×106 of MDA-MB-231 cells were subcutaneously injected into 7-week-old female Balb/c-nude mice. Four TGase inhibitors and doxorubicin were co-administered, and the effects were examined Group 1: saline. Group 2: Cisplatin (7.5 mg/kg). Group 3: 2-alkyloxy-3-phenylethynyl-4a,5-dihydropyrido[2,3-b]pyrazine derivative (4 mg/kg).

The prepared cancer cell, MDA-MB231/luciferase (8×106 cells/head) was subcutaneously injected into animals 7 days after their arrival. When a volume of the injected cancer cells reached 100 mm3, drug treatment was started. Cisplatin was given daily via i.p. injection for 5 days after initial treatment, and 2 days apart from this period, this schedule was repeated once more.

C. Studyal period. After injection of MDA-MB-231, when a volume of the injected tumor reached 100 mm3, drug treatment was performed. After 3-4 weeks, the mice were sacrificed.

D. Measurement of tumor size. After the tumor volume reached 100 mm3, the size was measured three times per week during the drug treatment period. The tumor volume was calculated as ‘long axis×long axis×short axis/2’.

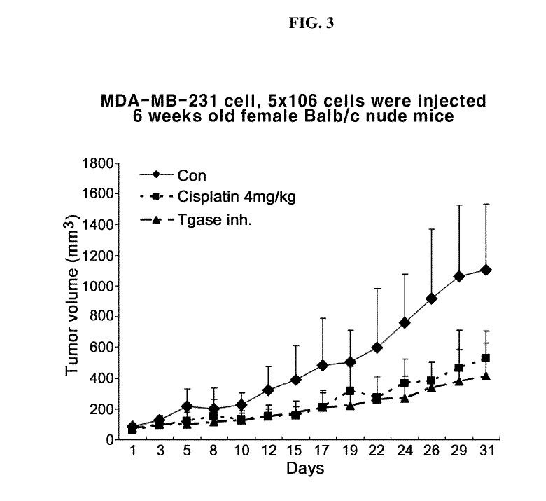

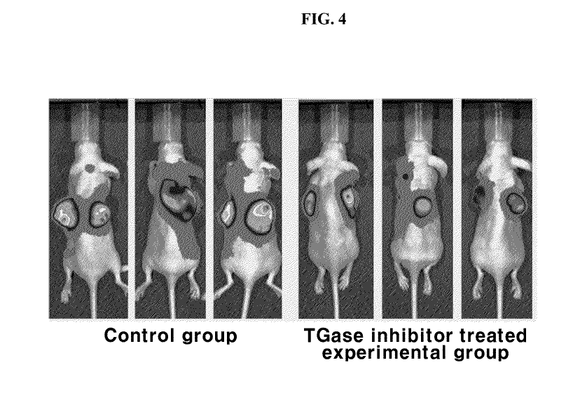

2) Result. The cisplatin-treated group showed 10% or more of weight loss due to toxicity. Further, the weight loss was not observed in the TGase 2 inhibitor-treated groups. Due to TGase 2 inhibition, a reduction in the tumor size was observed in the TGase 2 inhibitor-treated groups, compared to the cisplatin-treated group (FIGS. 3 and 4). Further, Example 1 among the 2-alkyloxy-3-phenylethynyl-4a,5-dihydropyrido[2,3-b]pyrazine derivative showed the best effect of a reduction in the tumor size.

Effect of the invention. The 2-alkyloxy-3-phenylethynyl-4a,5-dihydropyrido[2,3-b]pyrazine derivatives according to the present invention show excellent inhibitory effects on transglutaminase 2 activity.

What is claimed is:

1. A method for inhibiting transglutaminase 2 activity comprising contacting cells with an amount of a compound of the following Chemical Formula 1: 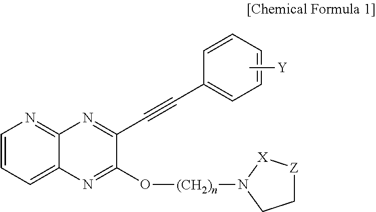
wherein Y is halogen, C1-4 alkoxy, C1-4 alkyl, C1-4 alkylamino or C1-4 haloalkyl;
X is —CO—, —CH2CH2- or —CH2-;
Z is —NH—, —O— or —CH2-; and
n is an integer of 1 to 3.

2. The method according to claim 1, wherein said X is —CO— and said Z is —NH— or —CH2-.

3. The method according to claim 1, wherein said X is —CH2CH2- and said Z is —O— or —CH2-.

4. The method according to claim 1, wherein said X is —CH2- and said Z is —CH2-.

5. The method according to claim 1, wherein said Y is halogen, methoxy, methyl, dimethylamino or trifluoroalkyl.

6. The method according to claim 1, wherein said n is 2 or 3.

7. The method according to claim 1, wherein the compound is any one selected from the group consisting of:
1) 3-(phenylethynyl)-2-(2-(pyrrolidin-1-yl)ethoxy)pyrido[3,2-b]pyrazine,
2) 3-(phenylethynyl)-2-(2-(piperidin-1-yl)ethoxy)pyrido[3,2-b]pyrazine,
3) 4-(2-(3-(phenylethynyl)pyrido[3,2-b]pyrazin-2-yloxy)ethyl)morpholine,
4) 3-(phenylethynyl)-2-(3-(pyrrolidin-1-yl)propoxy)pyrido[3,2-b]pyrazine,
5) 3-(phenylethynyl)-2-(3-(piperidin-1-yl)propoxy)pyrido[3,2-b]pyrazine,
6) 4-(3-(3-(phenylethynyl)pyrido[3,2-b]pyrazin-2-yloxy)propyl)morpholine,
7) 1-(3-(3-(phenylethynyl)pyrido[3,2-b]pyrazin-2-yloxy)propyl)pyrrolidin-2-one,
8) N,N-dimethyl-4-((2-(2-(pyrrolidin-1-yl)ethoxy)pyrido[3,2-b]pyrazin-3-yl)ethynyl)benzenamine,
9) N,N-dimethyl-4-((2-(2-(piperidin-1-yl)ethoxy)pyrido[3,2-b]pyrazin-3-yl)ethynyl)benzenamine,
10) 1-(2-(3-((4-(dimethylamino)phenyl)ethynyl)pyrido[3,2-b]pyrazin-2-yloxy)ethyl)pyrrolidin-2-one,
11) N,N-dimethyl-4-((2-(3-morpholinopropoxy)pyrido[3,2-b]pyrazin-3-yl)ethynyl)benzenamine,
12) 1-(3-(3-((4-(dimethylamino)phenyl)ethynyl)pyrido[3,2-b]pyrazin-2-yloxy)propyl)pyrrolidin-2-one,
13) 1-(2-(3-((4-(dimethylamino)phenyl)ethynyl)pyrido[3,2-b]pyrazin-2-yloxy)ethyl)imidazolidin-2-one,
14) 3-((4-methoxyphenyl)ethynyl)-2-(2-(pyrrolidin-1-yl)ethoxy)pyrido[3,2-b]pyrazine,
15) 3-((4-methoxyphenyl)ethynyl)-2-(2-(piperidin-1-yl)ethoxy)pyrido[3,2-b]pyrazine,
16) 1-(2-(3-((4-methoxyphenyl)ethynyl)pyrido[3,2-b]pyrazin-2-yloxy)ethyl)pyrrolidin-2-one,
17) 4-(2-(3-((4-methoxyphenyl)ethynyl)pyrido[3,2-b]pyrazin-2-yloxy)ethyl)morpholine,
18) 4-(3-(3-((4-methoxyphenyl)ethynyl)pyrido[3,2-b]pyrazin-2-yloxy)propyl)morpholine,
19) 4-(3-(3-((4-methoxyphenyl)ethynyl)pyrido[3,2-b]pyrazin-2-yloxy)propyl)morpholine,
20) 1-(2-(3-((4-methoxyphenyl)ethynyl)pyrido[3,2-b]pyrazin-2-yloxy)ethyl)imidazolidin-2-one,
21) 2-(2-(pyrrolidin-1-yl)ethoxy)-3-(p-tolylethynyl)pyrido[3,2-b]pyrazine,
22) 2-(2-(piperidin-1-yl)ethoxy)-3-(p-tolylethynyl)pyrido[3,2-b]pyrazine,
23) 1-(2-(3-(p-tolylethynyl)pyrido[3,2-b]pyrazin-2-yloxy)ethyl)pyrrolidin-2-one,
24) 4-(2-(3-(p-tolylethynyl)pyrido[3,2-b]pyrazin-2-yloxy)ethyl)molpholine,
25) 4-(3-(3-(p-tolylethynyl)pyrido[3,2-b]pyrazin-2-yloxy)propyl)morpholine,
26) 1-(3-(3-(p-tolylethynyl)pyrido[3,2-b]pyrazin-2-yloxy)propyl)pyaolidin-2-one,
27) 1-(2-(3-(p-tolylethynyl)pyrido[3,2-b]pyrazin-2-yloxy)ethyl)imidazolidin-2-one,
28) 2-(2-(pyrrolidin-1-yl)ethoxy)-3-((4-(trifluoromethyl)phenyl)ethynyl)pyrido[3,2-b]pyrazine,
29) 2-(2-(piperidin-1-yl)ethoxy)-3-((4-(trifluoromethyl)phenyl)ethynyl)pyrido[3,2-b]pyrazine,
30) 1-(2-(3-((4-(trifluoromethyl)phenyl)ethynyl)pyrido[3,2-b]pyrazin-2-yloxy)ethyl)pyrrolidin-2-one,
31) 4-(2-(3-((4-(trifluoromethyl)phenyl)ethynyl)pyrido[3,2-b]pyrazin-2-yloxy)ethyl)morpholine,
32) 4-(3-(3-((4-(trifluoromethyl)phenyl)ethynyl)pyrido[3,2-b]pyrazin-2-yloxy)propyl)morpholine,
33) 1-(3-(3-((4-(trifluoromethyl)phenyl)ethynyl)pyrido[3,2-b]pyrazin-2-yloxy)propyl)pyrrolidin-2-one,
34) 1-(2-(3-((4-(trifluoromethyl)phenyl)ethynyl)pyrido[3,2-b]pyrazin-2-yloxy)ethyl)imidazolidin-2-one,
35) N,N-dimethyl-3-((2-(2-(pyrrolidin-1-yl)ethoxy)pyrido[3,2-b]pyrazin-3-yl)ethynyl)benzenamine,
36) N,N-dimethyl-3-((2-(2-(piperidin-1-yl)ethoxy)pyrido[3,2-b]pyrazin-3-yl)ethynyl)benzenamine,
37) 2-(2-(3-((3-(dimethylamino)phenyl)ethynyl)pyrido[3,2-b]pyrazin-2-yloxy)ethyl)cyclopentanone,
38) N,N-dimethyl-3-((2-(2-morpholinoethoxy)pyrido[3,2-b]pyrazin-3-yl)ethynyl)benzenamine,
39) N,N-dimethyl-3-((2-(3-morpholinopropoxy)pyrido[3,2-b]pyrazin-3-yl)ethynyl)benzenamine,
40) 1-(3-(3-((3-(dimethylamino)phenyl)ethynyl)pyrido[3,2-b]pyrazin-2-yloxy)propyl)pyrrolidin-2-one,
41) 1-(2-(3-((3-(dimethylamino)phenyl)ethynyl)pyrido[3,2-b]pyrazin-2-yloxy)ethyl)imidazolidin-2-one,
42) N,N-dimethyl-2-((2-(2-(pyrrolidin-1-yl)ethoxy)pyrido[3,2-b]pyrazin-3-yl)ethynyl)benzenamine,
43) N,N-dimethyl-2-((2-(2-(piperidin-1-yl)ethoxy)pyrido[3,2-b]pyrazin-3-yl)ethynyl)benzenamine,
44) 1-(2-(3-((2-(dimethylamino)phenyl)ethynyl)pyrido[3,2-b]pyrazin-2-yloxy)ethyl)pyrrolidin-2-one,
45) N,N-dimethyl-2-((2-(2-morpholinoethoxy)pyrido[3,2-b]pyrazin-3-yl)ethynyl)benzenamine,
46) N,N-dimethyl-2-((2-(3-morpholinopropoxy)pyrido[3,2-b]pyrazin-3-yl)ethynyl)benzenamine,
47) 1-(3-(3-((2-(dimethylamino)phenyl)ethynyl)pyrido[3,2-b]pyrazin-2-yloxy)propyl)pyrrolidin-2-one,
48) 1-(2-(3-((2-(dimethylamino)phenyl)ethynyl)pyrido[3,2-b]pyrazin-2-yloxy)ethyl)imidazolidin-2-one,
49) 2-(2-(pyrrolidin-1-yl)ethoxy)-3-((3-(trifluoromethyl)phenyl)ethynyl)pyrido[3,2-b]pyrazine, and
50) 2-(2-(piperidin-1-yl)ethoxy)-3-((3-(trifluoromethyl)phenyl)ethynyl)pyrido[3,2-b]pyrazine.
